(19) United States
(12) Patent Application Publication
(10) Pub. No.: US 2014/0147807 A1
(43) Pub. Date: May 29, 2014

(54) COMPUTER-AIDED POSITIONING AND NAVIGATION SYSTEM FOR DENTAL IMPLANT

(71) Applicant: NATIONAL CHUNG CHENG UNIVERSITY, Chia-Yi (TW)

(72) Inventors: Hong-Tzong YAU, Chia-Yi County (TW); Yen-Kun LIN, New Taipei City (TW)

(73) Assignee: NATIONAL CHUNG CHENG UNIVERSITY, Chia-Yi (TW)

(21) Appl. No.: 13/777,464

(22) Filed: Feb. 26, 2013

(30) Foreign Application Priority Data
Nov. 27, 2012 (TW) 101144376

(51) Int. Cl. A61C 1/08; A61B 5/00; A61B 5/06; A61C 8/00

(52) U.S. Cl. CPC A61C 1/084; A61C 8/0089; A61B 5/0088; A61B 5/7425; A61B 5/064
USPC 433/173

(57) ABSTRACT

A computer-aided positioning and navigation system for dental implant includes a computer system having built therein a dental implant planning software and providing a 3D digital human tissues model to create an implant navigation information, a positioning assistive device including a body providing a positioning portion and a guide portion and a connection member carrying an optical positioning device, one or multiple optical capture devices, and a display device electrically connected to the computer system. The computer system controls the optical capture device to capture images and drives the display device to display a part of the content of the 3D digital human tissues model and the implant navigation information.

### BACKGROUND OF THE INVENTION

1. Field of the Invention

The present invention relates to dental implant technology and more particularly, to a computer-aided positioning and navigation system for dental implant.

2. Description of the Related Art

In dental implant technology, static and dynamic guide types of computer-aided positioning systems are known methods for accurate positioning to assist dental implant.

Static guide techniques, such as U.S. Pat. No. 5,725,376, U.S. Pat. No. 7,909,606 and U.S. Pat. No. 7,835,811, disclose the measure of performing image processing to extract the image of jaw bone tissues from the patient's 2D oral cavity image data that is obtained prior to dental implant, the measure of using a digital model reconstruction algorithm to reconstruct a digital model, the measure of loading in a planning software for planning a dental implant procedure and designing a positioning assistive device, and the final measure of making a positioning assistive device using a machining technique. This kind of positioning assistive device can provide a constant drilling direction physically during surgery. During surgery, this kind of positioning assistive device is to be positioned in the patient's mouth subjectively by the operator. Its position cannot be adjusted subject to the conditions of the patient's oral cavity. Further, the implanted depth must be repeatedly confirmed. More importantly, for a completely edentulous patient, the error will increase in case of inaccurate positioning of the dental surgical template.

Dynamic guide techniques, such as U.S. Pat. No. 6,640,128, U.S. Pat. No. 8,172,573 and U.S. Pat. No. 7,899,512, disclose the measure of performing image processing to extract the image of jaw bone tissues from the patient's 2D oral cavity image data that is obtained prior to dental implant, the measure of using a digital model reconstruction algorithm to reconstruct a digital model, the measure of loading in a planning software for planning a dental implant procedure, and the final measure of performing positioning of virtual implant on the real jaw bone of the patient. Subject to assistance of an optical positioning device or the display on a display screen, the dentist can adjust the positioning of the implant based on the visual feedback. However, due to lack of a dental surgical template or other physical guiding element, these dynamic guide techniques cannot provide dentists with a physical means to guide the drilling position and direction with accuracy and consistency.

In conclusion, the currently known techniques are unable to provide an accurate and stable dental implant surgical procedure, and dentists can only choose one of the static or dynamic guide techniques mentioned above, so, there is no robust solution at the present time.

Further, from the above prior art techniques, we can see that any positioning assistive technique employed during dental implant procedure is not human surgery technology itself; it is simply a supplementary technique to assist positioning, and therefore it should not be contrary to the provisions of the Patent Law.

### SUMMARY OF THE INVENTION

The present invention has been accomplished under the circumstances in view. It is the main object of the present invention to provide a computer-aided positioning and navigation system Which uses a positioning assistive device to guide drilling in a constant direction physically and enables 3D navigation by displaying the patient's 3D digital human tissue model and the related implant navigation information on a display device so that the dentist can adjust and verify the drilling operation dynamically, enhancing the accuracy and safety of the dental implant operation.

To achieve this and other objects of the present invention, a computer-aided positioning and navigation system comprises a computer system, a positioning assistive device, at least one optical capture device, and a display device. The computer system has installed therein a dental implant planning software. The computer system is adapted to load in a two-dimensional digital oral cavity image data obtained from the patient, to convert the two-dimensional digital oral cavity image data into a three-dimensional digital human tissues model, and to create an implant navigation information by finding an optimal navigation method using the 3D human tissues model. The positioning assistive device is made by using the 3D digital human tissues model to design a digital structure and then actually creating an entity structure subject to the digital structure for use as the positioning assistive device, so that, the spatial relationship between the digital structure and the 3D digital human tissues model resembles the spatial relationship between the positioning assistive device and the patient. The positioning assistive device comprises a body and a connection member extending outwardly from said body. The body comprises a positioning portion, and at least one guide portion. The positioning portion is adapted to position the body in the patient's oral cavity for enabling the at least one guide portion to be held inside the patient's oral cavity. The at least one guide portion is adapted to guide the implant direction. The connection member is suspended outside the patient's oral cavity and carrying thereon an optical positioning device. The body and the optical positioning device create a first spatial relationship therebetween. The at least one optical capture device is electrically connected to the computer system. The display device is also electrically connected to the computer system. The computer system drives the optical capture device to capture images containing at least the images of the patient's mouth and the optical positioning device. After image pickup, the computer system computes a second spatial relationship between the optical positioning device and the optical capture device, and then displays at least a part of the content of the 3D digital human tissues model on the display device subject to the second spatial relationship and the first spatial relationship, and also displays the implant navigation information on the display device.

Preferably, the optical positioning device is affixed to the connection member by means of at least one fastening member.

Preferably, the first spatial relationship and the second spatial relationship both contain distance and angle, and both are different.

Preferably, the implant navigation information contains at least one drilling location mark, at least one drill pilot line or the hardness of the bone at each drilling location, or their combinations. The at least one drill pilot line corresponds to the at least one guide portion.

Preferably, the at least one guide portion each is a through hole formed on the body.

Preferably, the display device is a display screen. The computer system controls the display device to display the 3D digital human tissues model and the implant navigation information and photographed images around the patient's mouth. The 3D digital human tissues model and the implant navigation information are superimposed on the photographed images around the patient's mouth.

Preferably, the display device is a head-mounted display comprising a transparent display screen. The at least one optical capture device is mounted at the head-mounted display. Subject to the transparent characteristic of the transparent display screen, the wearer wearing the head-mounted display sees through the display screen and watches the patient's mouth. When the computer system displays the 3D digital human tissues model and the implant navigation information on the display device, the images are superimposed over the line of sight of the wearer viewing the patient's mouth, enabling the wearer to feel the 3D digital human tissues model and the implant navigation information are superimposed on the patient's mouth.

Preferably, the content displayed by the computer system on the display screen is changed at any time with the change in the position or angle of the head of the wearer, enabling the wearer to feel the 3D digital human tissues model and the implant navigation information are constantly superimposed on the patient's mouth.

Preferably, the number of the at least one optical capture device is 2, and these two optical capture devices are disposed at different positions and angles relative to the optical positioning device.

Preferably, the two optical capture devices are arranged at right angles, or disposed to simulate the angles and positions of the eyes of a human being.

Preferably, the computer-aided positioning and navigation system further comprises a switch electrically connected to the computer system and operable to switch the displayed content of the display device between the 3D digital human tissues model and the implant navigation information.

Preferably, the switch is selected from the group of foot switches, touch switches, pushbutton switches and voice-activated switches.

Preferably, the first spatial relationship is constant when created.

Preferably, the implant navigation information contains a drill stop. The computer system further comprises a drill stop matching procedure. The drill stop matching procedure uses the images captured by the at least one optical capture device to match a particular characteristic of the tool being operated by the dentist to be superimposed on the drill stop or not.

Preferably, the drill stop matching procedure gives off an alarm signal if the particular characteristic of the tool being operated by the dentist is superimposed on the drill stop. The alarm signal can be a video alarm signal, or an audio alarm signal.

Other advantages and features of the present invention will be fully understood by reference to the following specification in conjunction with the accompanying drawings, in which like reference signs denote like components of structure.

### BRIEF DESCRIPTION OF THE DRAWINGS

FIG. 1 illustrates the architecture of a computer-aided positioning and navigation system in accordance with a first embodiment of the present invention.

FIG. 2 is a block diagram of a part of the computer-aided positioning and navigation system in accordance with the first embodiment of the present invention.

FIG. 3 is an elevational view of a part of the computer-aided positioning and navigation system in accordance with the first embodiment of the present invention, illustrating the structure of the positioning assistive device.

FIG. 4 is a schematic installed view of the positioning assistive device of the computer-aided positioning and navigation system in accordance with the first embodiment of the present invention.

FIG. 5 is a schematic operational view of the computer-aided positioning and navigation system in accordance with the first embodiment of the present invention, illustrating the relationship between the optical capture device and the optical positioning device.

FIG. 6 is another schematic operational view of the computer-aided positioning and navigation system in accordance with the first embodiment of the present invention, illustrating the 3D digital human tissues model and the implant navigation information displayed on the display device.

FIG. 7 is still another schematic operational view of the computer-aided positioning and navigation system in accordance with the first embodiment of the present invention, illustrating the images photographed by the optical capture device displayed on the display device.

FIG. 8 is still another schematic operational view of the computer-aided positioning and navigation system in accordance with the first embodiment of the present invention, illustrating the images photographed by the optical capture device, the 3D digital human tissues model and the implant navigation information displayed on the display device.

FIG. 9 illustrates the architecture of a computer-aided positioning and navigation system in accordance with a second embodiment of the present invention.

FIG. 10 is a block diagram of the computer system of the computer-aided positioning and navigation system in accordance with the second embodiment of the present invention.

FIG. 11 is an elevational view of a part of the computer-aided positioning and navigation system in accordance with the second embodiment of the present invention, illustrating the arrangement of the display device and the optical capture device.

FIG. 12 is a schematic applied view of the computer-aided positioning and navigation system in accordance with the second embodiment of the present invention.

FIG. 13 is a schematic operational view of the computer-aided positioning and navigation system in accordance with the second embodiment of the present invention, illustrating the images photographed by the optical capture device, the 3D digital human tissues model and the implant navigation information displayed on the display device.

### DETAILED DESCRIPTION OF THE INVENTION

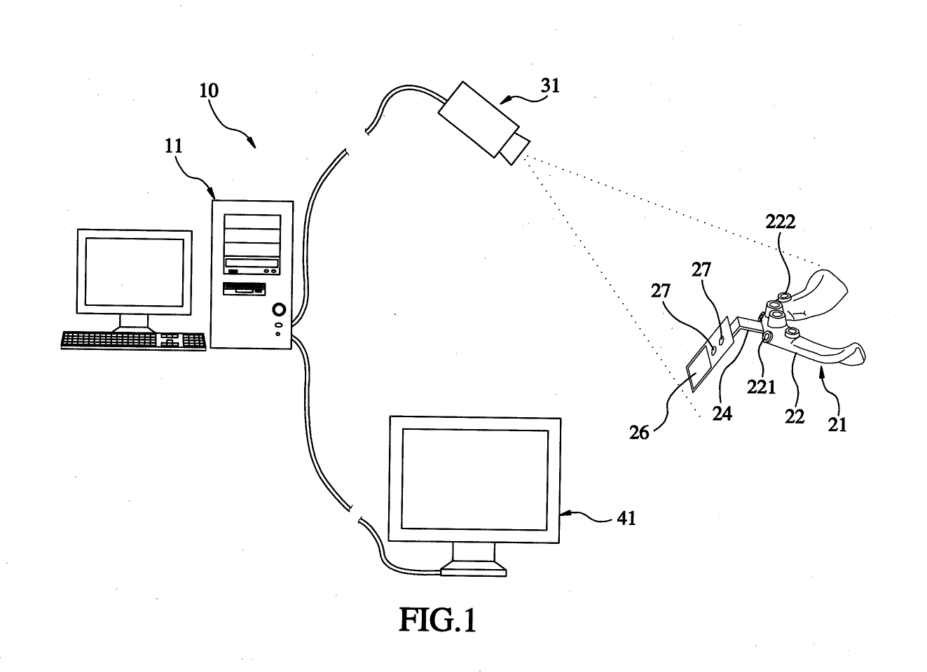

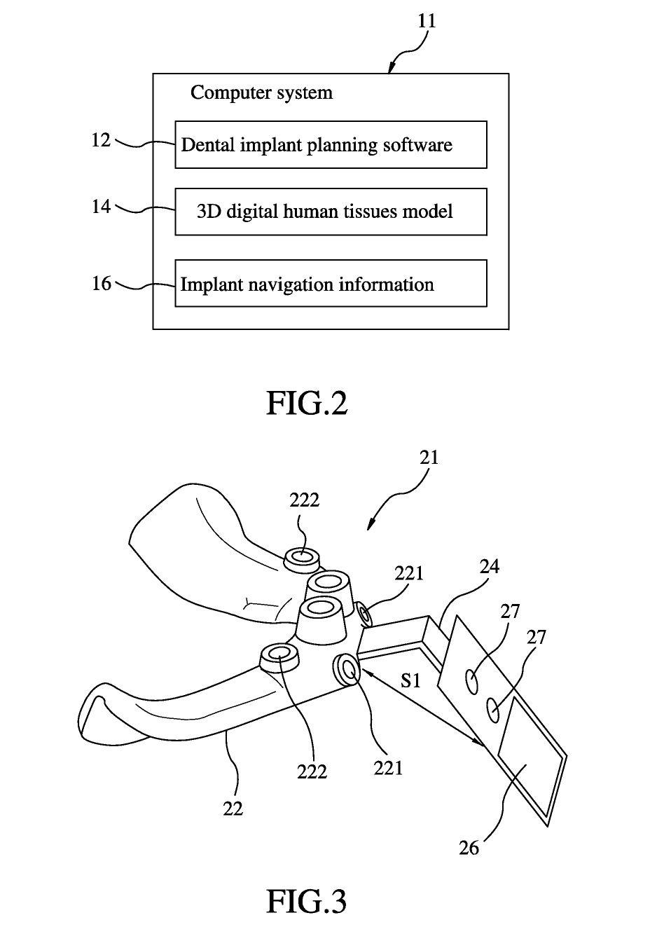

Referring to FIGS. 1-7, a computer-aided positioning and navigation system for dental implant in accordance with a first embodiment of the present invention is shown. The computer-aided positioning and navigation system 10 comprises a computer system 11, a positioning assistive device 21, an optical capture device 31, and a display device 41.

The computer system 11 has installed therein a dental implant planning software 12, and is adapted to load in a two-dimensional digital oral cavity image data being obtained from the patient, to convert the two-dimensional digital oral cavity image data into a 3D digital human tissues model 14, and to create an implant navigation information 16 by finding the optimal navigation method using the 3D digital human tissues model 14. The dental implant planning software 12 is of the known technique, no further description is needed.

The positioning assistive device 21 is made by: using the 3D digital human tissues model 14 to design a digital structure (not shown) and then actually creating an entity structure subject to the digital structure for use as the positioning assistive device 21. Thus, the spatial relationship between the digital structure and the 3D digital human tissues model 14 resembles the spatial relationship between the positioning assistive device 21 and the patient. The positioning assistive device 21 comprises a body 22, and a connection member 24 extending outwardly from the body 22. The body 22 comprises a positioning portion 221, and at least one guide portion 222. The positioning portion 221 is adapted to position the body 22 in the patient's oral cavity, enabling the at least one guide portion 222 to be held inside the patient's oral cavity. In this embodiment, the body 22 comprises a plurality of guide portions 222 adapted to guide the implant direction. The connection member 24 suspends outside the patient's oral cavity, carrying thereon an optical positioning device 26. Thus, a first spatial relationship S1 is created between the body 22 and the optical positioning device 26. This first spatial relationship S1 is constant.

In this embodiment, the number of the aforesaid at least one optical capture device 31 is 1. Further, this optical capture device 31 is electrically connected to the computer system 11. In this embodiment, the optical capture device 31 is a video camera.

The display device 41 is electrically connected to the computer system 11. In this embodiment, the display device 41 is a screen.

The computer system 11 can capture images by means of the optical capture device 31. The capture range includes at least the patient's mouth as well as the optical positioning device 26. After capturing image, the computer system 11 computes a second spatial relationship S2 between the optical positioning device 26 and the optical capture device 31, and then displays at least a part of the content of the 3D digital human tissues model 14 on the display device 41 subject to the second spatial relationship S2 and the first spatial relationship S1, and also displays the implant navigation information 16 on the display device 41. When displaying the 3D digital human tissues model 14, it can simply display the nasal sinuses and nerve canals, i.e., display a part of the content of the 3D digital human tissues model 14.

It is to be noted that, in this embodiment, the optical positioning device 26 is affixed to the connection member 24 by two fastening members 27, for example, screws. Alternatively, the optical positioning device 26 can be integrally made with the body 22 and the connection member 24 in one piece, eliminating the use of the two fastening members 27 to affix the optical positioning device 26 to the connection member 24.

Further, in this embodiment, the first spatial relationship S1 and the second spatial relationship S2 both contain distance and angle. Further, the first spatial relationship S1 and the second spatial relationship S2 are different.

Further, in this embodiment, the implant navigation information 16 contains a plurality of drilling location marks, a plurality of drill pilot lines or the hardness of the bone at each drilling location, or their combinations. In the case that the implant navigation information 16 contains drill pilot lines, each drill pilot line corresponds to one respective guide portion 222.

Further, the guide portions 222 in this embodiment are through holes formed on the e body 22. In another embodiment of the present invention, each guide portion 222 can be formed by embedding one respective tube in the body 22.

In this embodiment, the computer system 11 controls the display device 41 to display the 3D digital human tissues model 14 and the implant navigation information 16 as well as the images actually photographed around the patient's mouth. The 3D digital human tissues model 14 and the implant navigation information 16 are superimposed on the images around the patient's mouth. Thus, if the nasal sinuses are displayed when the dentist and other dental professionals are viewing the superimposed 3D digital human tissues model 14, the dentist and other dental professionals can see the locations of the nasal sinuses at patient's face, and thus, the displayed content can assist the dentist not to drill into the nasal sinuses during dental implant drilling. If the nerves canal is displayed, the displayed content can assist the dentist not to drill into the nerves canal during dental implant drilling, avoiding postoperative complications.

After understanding of the architecture of the first embodiment of the present invention, the operation of the first embodiment of the present invention will be described hereinafter.

Before the operation of the present invention, it is necessary to establish the patient's 3D digital human tissues model 14 and then to create the corresponding implant navigation information 16.

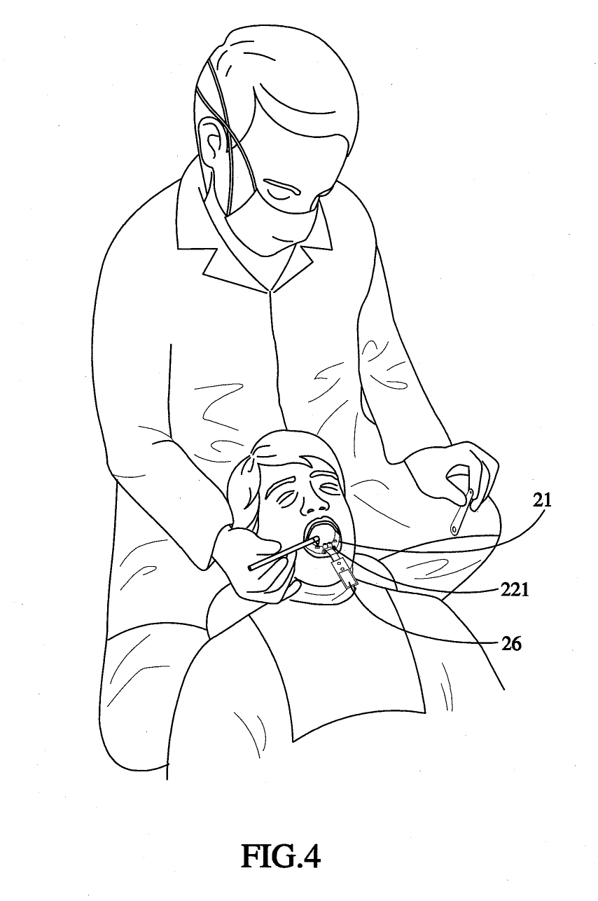

Referring to FIG. 4, when operating the computer-aided positioning and navigation system, position the positioning assistive device 21 in the patient's oral cavity by means of the positioning portion 221. Positioning the positioning assistive device 21 in the patient's oral cavity can be done by inserting screws (not shown) through the positioning portion 221 and then driving the screws into the bone in the patient's oral cavity. Further, the positioning portion 221 can be shaped like a cap and press-fitted onto the patient's healthy teeth. After positioning of the positioning assistive device 21 in the patient's oral cavity, the optical positioning device 26 must be kept outside the patient's oral cavity.

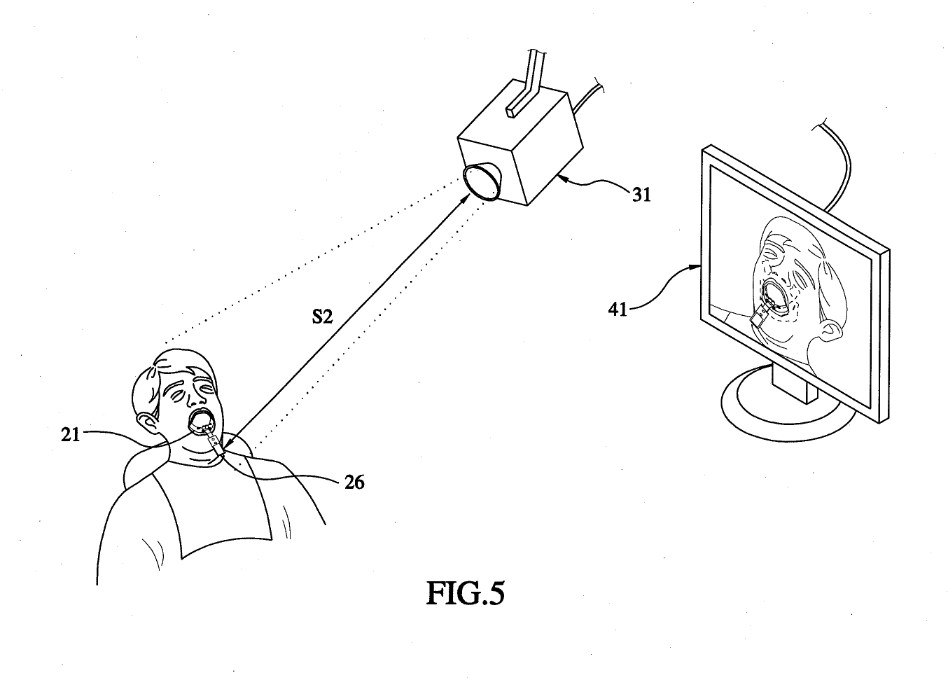

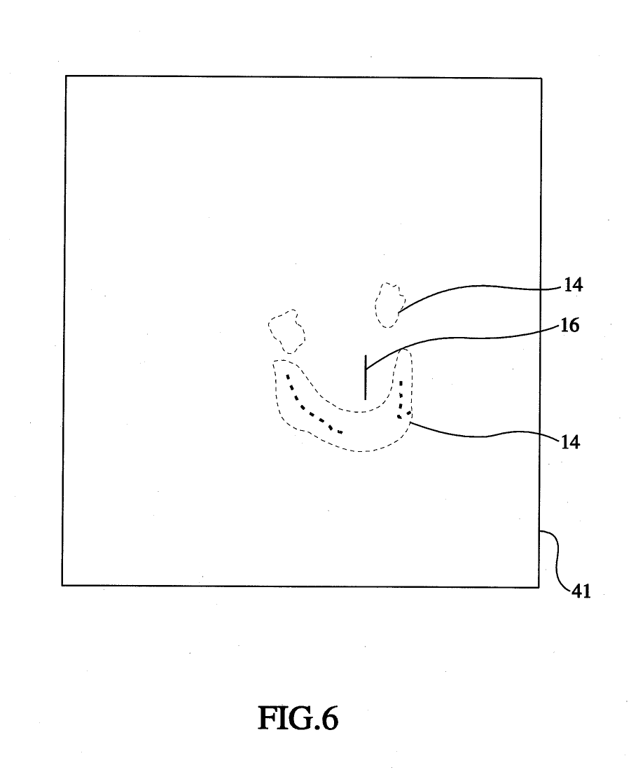

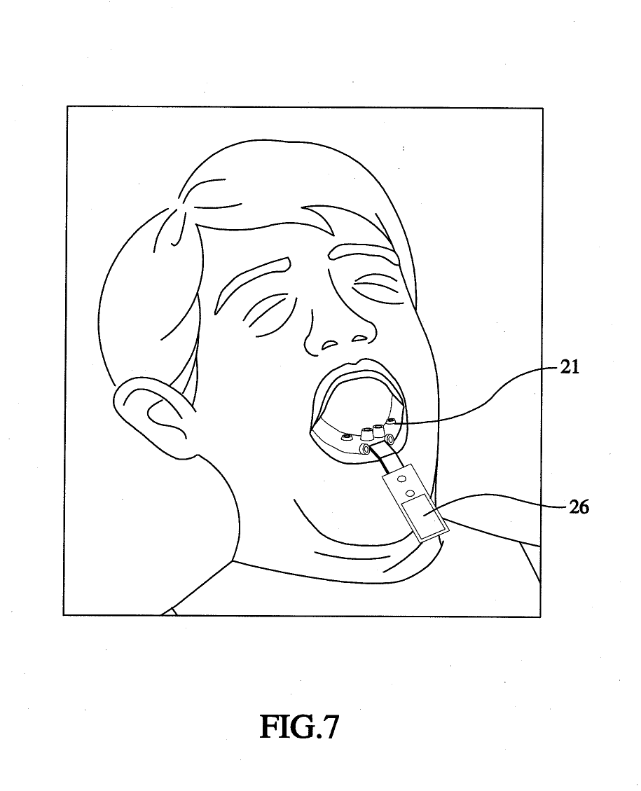

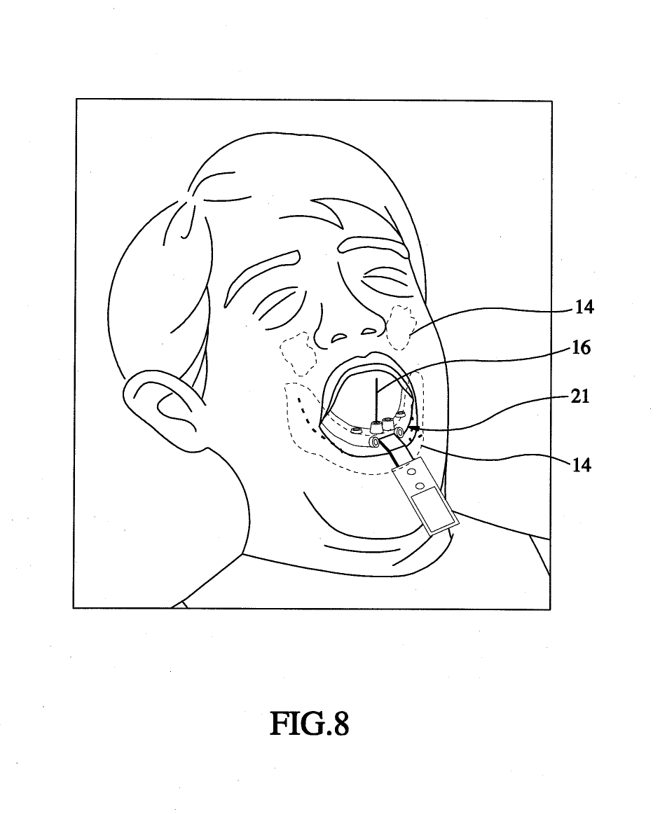

Referring to FIGS. 5-8, use the optical capture device 31 to capture images, enabling the computer system 11 to display the captured images on the display device 41. Further, the computer system 11 will also calculate the second spatial relationship S2, thereby obtaining the spatial relationship between the optical capture device 31 and the optical positioning device 26. Thus, the computer system 11 can display the 3D digital human tissues model 14 and the implant navigation information 16 on the display device 41 by means of the second spatial relationship S2 and the first spatial relationship S1. At the same time, the computer system 11 can also display on the display device 41 the images of the patient's oral cavity (including the images of the dentist's hands and related tools). For example, if the implant navigation information 16 is drill pilot lines, the dentist will see the drill pilot lines on the display device 41, and can then compare the locations of the drill pilot lines in the images to the tool being held in the hand so as to make sure of the correctness of the drilling direction. Further, because the patient's 3D digital human tissues model 14 is displayed, the computer system 11 can be controlled to selectively display key body tissues of the patient around the oral cavity, such as nasal sinuses or nerves canal. By means of viewing the displaying content, the dentist can understand the depth or location of the current drilling is still safe for the patient. During display, FIG. 6 illustrates the status of the 3D digital human tissues model 14 and the implant navigation information 16 displayed on the display device 41; FIG. 7 illustrates the images of the patient's face displayed on the display device 41; FIG. 8 illustrates the content of FIG. 6 and the content of FIG. 7 displayed on the display device 41.

Further, because the positioning assistive device 21 comprises a plurality of guide portions 222 that provide the function of guide holes of a conventional dental implant template, the guide portions 222 can guide the drilling direction when the dentist performs drilling. By means of the drill stop design of the guide portions 222, the drilling tool can be stopped when reached a certain depth. However, this drill stop design is of the known art and commonly seen in conventional dental implant template, no further detailed description in this regard will be necessary.

In general, the first embodiment of the present invention uses the positioning assistive device 21 to guide drilling in a constant direction physically, and enables the patient's 3D digital human tissues model 14 and the implant navigation information 16 to be displayed on the display device 41 so that the dentist can adjust the drilling operation dynamically, enhancing the accuracy and safety of the dental implant operation.

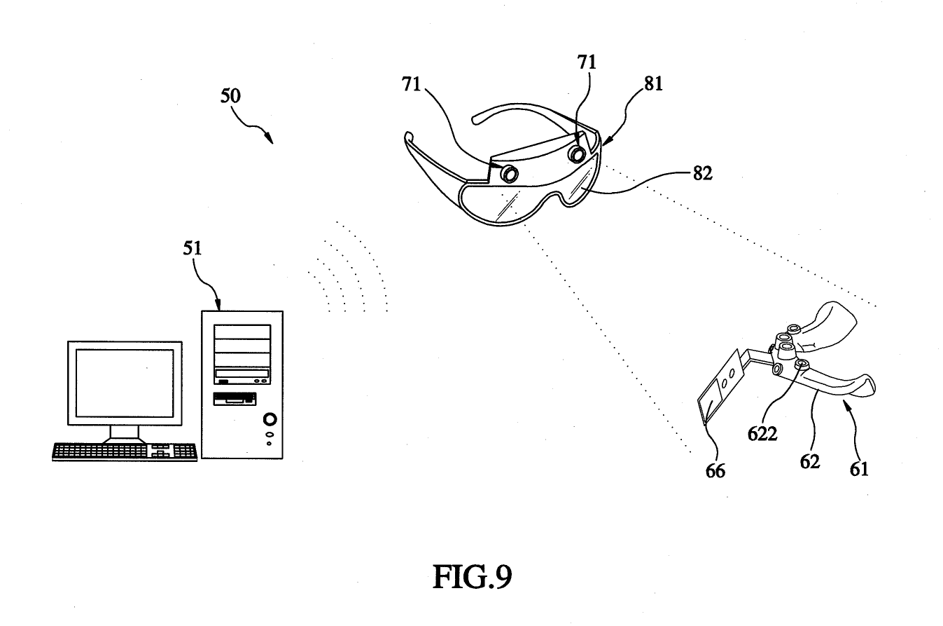

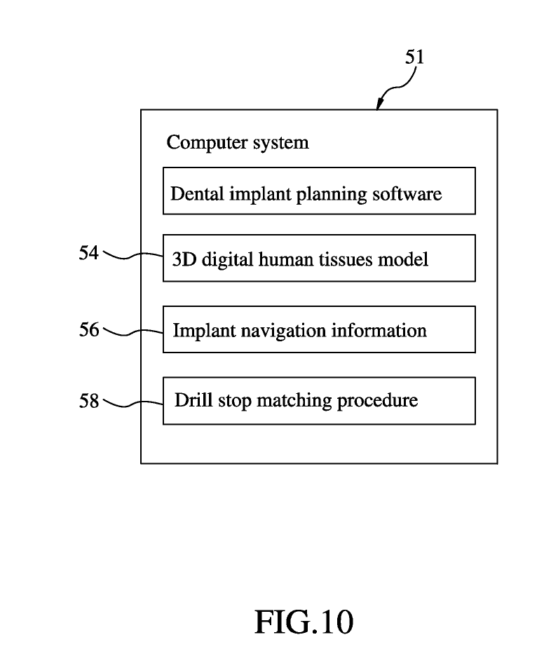

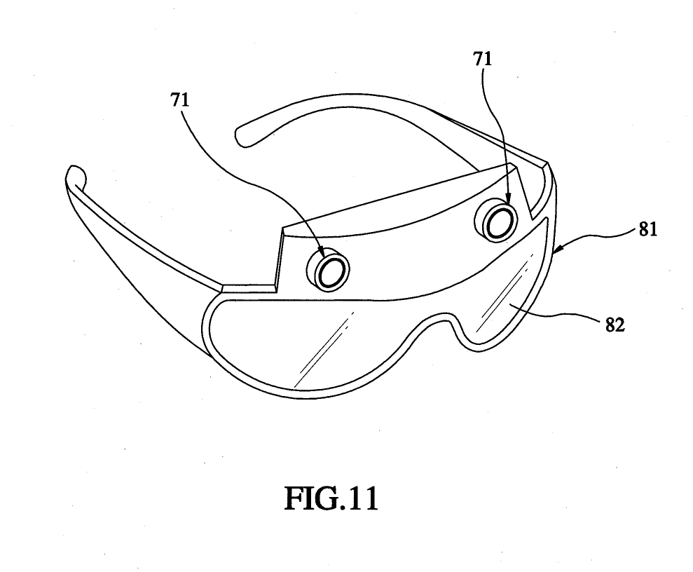

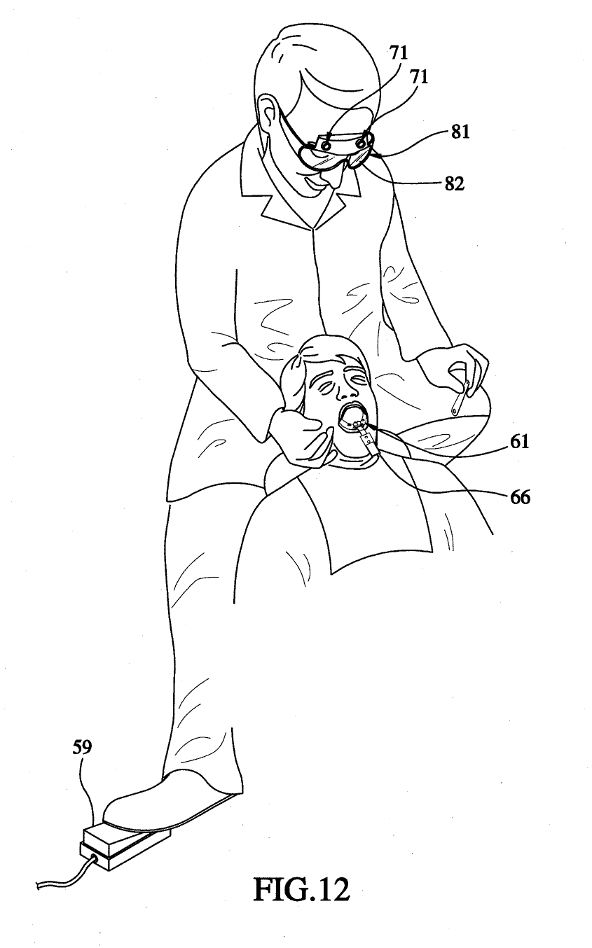

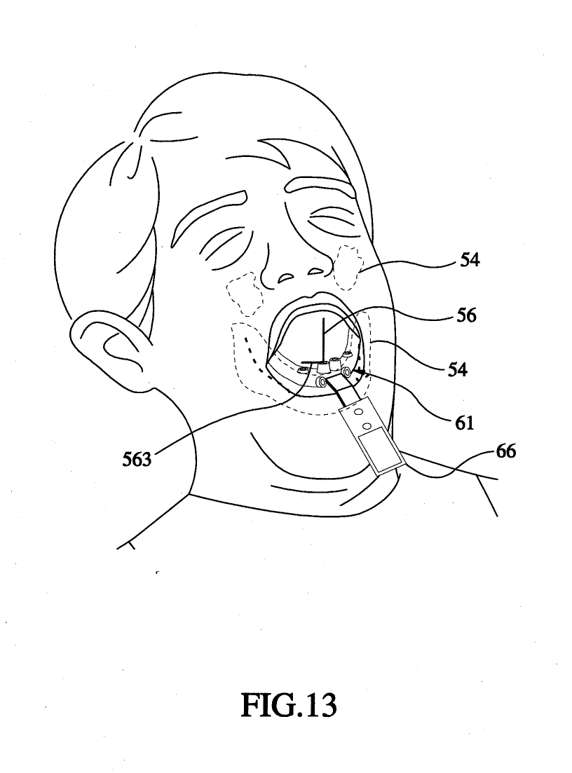

FIGS. 9-11 illustrate a computer-aided positioning and navigation system 50 in accordance with a second embodiment of the present invention. This second embodiment is substantially similar to the aforesaid first embodiment with the exceptions stated below.

The number of the at least one optical capture device 71 is 2. These two optical capture devices 71 are disposed at different positions and angles relative to the optical positioning device 66. For example, these two optical capture devices 71 are arranged at right angles or to simulate the angles and positions of the eyes of a human being, so that two pictures can be displayed on the display device 81 at different angles, enabling the dentist to accurately determine the implant navigation information 56.

The display device 81 is a HMD (head-mounted display). The display device 81 comprises a transparent display screen 82 that enables the user to see the real scene outside the transparent display screen 82 as well as the displayed content.

The two optical capture devices 71 are mounted on the display device 81.

The wearer (dentist) can see through the transparent display screen 82 and watch the patient's mouth. When the computer system 51 displays the 3D digital human tissues model 54 and the implant navigation information 56 on the display device 81, the images are superimposed over the line of sight of the wearer viewing the patient's mouth, enabling the wearer to feel the 3D digital human tissues model 54 and the implant navigation information 56 are superimposed on the patient's mouth. Unlike the aforesaid first embodiment, the display device 81 of this second embodiment does not display the images of the patient photographed by the optical capture devices 71. The transparent characteristic of the display screen 82 admits the wearer's line of sight, enabling the wearer to see the body of the patient directly. Further, the content displayed by the computer system 51 on the display screen 82 can be changed at any time with the change in the position or angle of the head of the wearer, enabling the wearer to feel the 3D digital human tissues model 54 and the implant navigation information 56 are constantly superimposed on the patient's mouth.

This second embodiment further comprises a switch 59 electrically connected to the computer system 51 for switching the displayed content of the display device 81 between the 3D digital human tissues model 54 or the implant navigation information 56. The switch 59 can be a foot switch, a touch switch, a pushbutton switch, or a voice-activated switch. Thus, the dentist can selectively drive the display device 81 to display the 3D digital human tissues model 54 or the implant navigation information 56 by means of the switch 59, so that the dentist can see the part shielded by the 3D digital human tissues model 54 or the implant navigation information 56.

Thus, the second embodiment of the present invention enables the wearer (dentist) to see through the display device 81 and watch the patient's mouth. Further, the 3D digital human tissues model 54 and the implant navigation information 56 viewed by the wearer are constantly superimposed on the patient's mouth to guide the drilling direction during dental implant drilling. Further, the guide portion 622 at the body 62 of the positioning assistive device 61 provides a physical function to guide drilling in a constant direction.

Further, in this second embodiment, the implant navigation information 56 further comprises a drill stop 563; the computer system 51 further comprises a drill stop matching procedure 58. This drill stop matching procedure 58 uses the images captured by the optical capture device 71 to match a particular characteristic of the tool being operated by the dentist (for example, the bottom of the head member of the tool) to be superimposed on the drill stop 563 or not. If a superimposed condition is matched, an alarm signal is provided. This alarm signal can be a video alarm signal, or an audio alarm signal. Thus, this drill stop matching procedure assists the dentist to determine whether or not the drilling has reached the drill stop, preventing over-drilling.

The other structural details and effects of this second embodiment are same as the aforesaid first embodiment, and shall not be further described.

1. A computer-aided positioning and navigation system for dental implant, comprising:
a computer system having installed therein a dental implant planning software, said computer system being adapted to load in a two-dimensional digital oral cavity image data being obtained from the patient, to convert said two-dimensional digital oral cavity image data into a 3D digital human tissues model, and to create an implant navigation information by finding an optimal navigation method using said 3D digital human tissues model;
a positioning assistive device made by using said 3D digital human tissues model to design a digital structure and then actually creating an entity structure subject to said digital structure for use as the positioning assistive device, so that, the spatial relationship between said digital structure and said 3D digital human tissues model resembles the spatial relationship between said positioning assistive device and the patient, said positioning assistive device comprising a body and a connection member extending outwardly from said body, said body comprising a positioning portion and at least one guide portion, said positioning portion being adapted to position said body in the patient's oral cavity for enabling said at least one guide portion to be held inside the patient's oral cavity, said at least one guide portion being adapted to guide the implant direction, said connection member being suspended outside the patient's oral cavity and carrying thereon an optical positioning device, said body and said optical positioning device creating a first spatial relationship therebetween;
at least one optical capture device electrically connected to said computer system; and
a display device electrically connected to said computer system;
wherein said computer system drives said optical capture device to capture images containing at least the images of the patient's mouth and said optical positioning device; after capturing image, said computer system computes a second spatial relationship between said optical positioning device and said optical capture device, and then displays at least a part of the content of said 3D digital human tissues model on said display device subject to said second spatial relationship and said first spatial relationship, and also displays said implant navigation information on said display device.

2. The computer-aided positioning and navigation system as claimed in claim 1, wherein said optical positioning device is affixed to said connection member by means of at least one fastening member.

3. The computer-aided positioning and navigation system as claimed in claim 1, wherein said first spatial relationship and said second spatial relationship both contain distance and angle, and both are different.

4. The computer-aided positioning and navigation system as claimed in claim 1, wherein said implant navigation information contains at least one drilling location mark, at least one drill pilot line or the hardness of the bone at each drilling location, or their combinations, said at least one drill pilot line corresponding to said at least one guide portion.

5. The computer-aided positioning and navigation system as claimed in claim 4, wherein said at least one guide portion each is a through hole formed on said body.

6. The computer-aided positioning and navigation system as claimed in claim 1, wherein said display device is a display screen; said computer system controls said display device to display said 3D digital human tissues model and said implant navigation information and photographed images around the patient's mouth, said 3D digital human tissues model and said implant navigation information being superimposed on the photographed images around the patient's mouth.

7. The computer-aided positioning and navigation system as claimed in claim 1, wherein said display device is a head-mounted display comprising a transparent display screen; said at least one optical capture device is mounted at said head-mounted display; subject to the transparent characteristic of said transparent display screen, the wearer wearing said head-mounted display sees through said display screen and watches the patient's mouth; when said computer system displays said 3D digital human tissues model and said implant navigation information on said display device, the images are superimposed over the line of sight of the wearer viewing the patient's mouth, enabling the wearer to feel said 3D digital human tissues model and said implant navigation information are superimposed on the patient's mouth.

8. The computer-aided positioning and navigation system as claimed in claim 7, wherein the content displayed by said computer system on said display screen is changed at any time with the change in the position or angle of the head of the wearer, enabling the wearer to feel said 3D digital human tissues model and said implant navigation information are constantly superimposed on the patient's mouth.

9. The computer-aided positioning and navigation system as claimed in claim 1, wherein the number of said at least one optical capture device is 2, and these two said optical capture devices are disposed at different positions and angles relative to said optical positioning device.

10. The computer-aided positioning and navigation system as claimed in claim 9, wherein said two optical capture devices are arranged at right angles or to-simulate the angles and positions of the eyes of a human being.

11. The computer-aided positioning and navigation system as claimed in claim 1, further comprising a switch electrically connected to said computer system and operable to switch the displayed content of said display device between said 3D digital human tissues model or said implant navigation information.

12. The computer-aided positioning and navigation system as claimed in claim 11, wherein said switch is selected from the group of foot switches, touch switches, pushbutton switches and voice-activated switches.

13. The computer-aided positioning and navigation system as claimed in claim 1, wherein said first spatial relationship is constant when created.

14. The computer-aided positioning and navigation system as claimed in claim 1, wherein said implant navigation information contains a drill stop; said computer system further comprises a drill stop matching procedure, said drill stop matching procedure using the images captured by said at least one optical capture device to match a particular characteristic of the tool being operated by the dentist to be superimposed on said drill stop or not.

15. The computer-aided positioning and navigation system as claimed in claim 1, wherein said drill stop matching procedure gives off an alarm signal if said particular characteristic of the tool being operated by the dentist is superimposed on said drill stop, said alarm signal being a video alarm signal or an audio alarm signal.
